(19) United States
(12) Patent Application Publication
(10) Pub. No.: US 2010/0310494 A1
(43) Pub. Date: Dec. 9, 2010

(54) Styrax Benzoin Rejuvenating Cosmetic Preparation

(76) Inventor: Anushya Sundaresan, Pinner (GB)

Correspondence Address: Anushya;Sundaresan, Fairfield, Oxhey Lane, Pinner, HA5 4AN, omitted

(21) Appl. No.: 12/677,822

(22) PCT Filed: Nov. 10, 2008
(86) PCT No.: PCT/GB08/03788

§ 371 Date: Mar. 12, 2010

(51) Int. Cl. A61K 31/78; A61K 31/235; A61Q 19/00

(52) U.S. Cl. 424/78.03; 514/544

(57) ABSTRACT

A Styrax Benzoin Rejuvenating Cosmetic Preparation for immediate noticeable beneficial results on whole body, face and skin, embodying chemical constituents comprising, Benzoic acid, Cinnamic Acid, Benzoic Aldehyde, Vanillin Aldehyde, Benzyl Benzoate, and optionally, as well as additionally, a face cream base which includes Glycerine, Triglyceride, Polyacrylate, Phenoxyethamol, Methylparaben, Ethylparaben & Water.

### FIELD OF THE INVENTION

The present invention relates to a Styrax Benzoin Rejuvenating Cosmetic Preparation, supplied in liquid, lotion and cream form, together with dispensers and packaging, for immediate noticeable beneficial results externally on whole body, face and skin.

Cosmetic Uses & Benefits:

Skin: Initial beneficial results show preparation to give skin a youthful glow.

### EXISTING STATE OF THE ART

Although numerous skin creams have been available over the years, the applicant believes that none of them possess the versatility or immediate noticeable beneficial results of the present invention.

Prior Art—Patent & Product Searches—No Anticipations

At the time of filing, a Worldwide Patent Database Search carried out on the European Patent Office esp@cenet website showed no prior art of a Cosmetic Preparation, embodying the chemical constituents cited in the claims of the present invention.

Also, Product Searches on the World Wide Web showed no product anticipations.

### SUMMARY OF INVENTION

A Styrax Benzoin Rejuvenating Cosmetic Preparation for immediate noticeable beneficial results on skin, embodying chemical constituents comprising Benzoic acid, Cinnamic Acid, Benzoic Aldehyde, Vanillin Aldehyde, Benzyl Benzoate and optionally, as well as additionally, a face cream base which includes Glycerine, Triglyceride, Polyacrylate, Phenoxyethamol, Methylparaben, Ethylparaben & Water.

### COSMETIC USES & BENEFITS

1. Gives skin an immediate noticeable beneficial youthful glow.

### DESCRIPTION

1. The present invention consists of a Styrax Benzoin Rejuvenating Cosmetic Preparation supplied in liquid, lotion and cream form, together with dispensers packaging means, for immediate noticeable beneficial results on whole body face and skin, embodying chemical constituents comprising:
a. Benzoic acid,
b. Cinnamic Acid,
c. Benzoic Aldehyde,
d. Vanillin Aldehyde,
e. Benzyl Benzoate (Ester) and optionally as well as additionally,
f. A face cream base which includes Glycerine, Triglyceride, Polyacrylate, Phenoxyethamol, Methylparaben, Ethylparaben & Water.

2. A Styrax Benzoin Rejuvenating Cosmetic Preparation in liquid, lotion and cream form, wherein the dispensers include plastic bottle dispensers, ball applicator dispensers, pump action dispensers and squeeze tube dispensers.

1. A Styrax Benzoin Rejuvenating Cosmetic Preparation, supplied in liquid, lotion and cream form, together with dispensers and packaging means, for immediate noticeable beneficial use on whole body, face and skin, embodying chemical constituents comprising:
a. Benzoic acid,
b. Cinnamic Acid,
c. Benzoic Aldehyde,
d. Vanillin Aldehyde,
e. Benzyl Benzoate (Ester) and optionally as well as additionally,
f. A face cream base which includes Glycerine, Triglyceride, Polyacrylate, Phenoxyethamol, Methylparaben, Ethylparaben & Water.

2. A Styrax Benzoin Rejuvenating Cosmetic Preparation as in claim 1, wherein said dispensers and packaging means includes plastic bottle dispensers, ball applicator dispensers, pump action dispensers and squeeze tube dispensers.
